(19) United States
(12) Patent Application Publication
(10) Pub. No.: US 2025/0188474 A1
(43) Pub. Date: Jun. 12, 2025

(54) TREATMENT AND DIAGNOSIS OF INFLAMMATORY DISORDERS

(71) Applicants: Zhenglun Zhu, Newton, MA (US); Hong Gao, Dowingtown, PA (US)

(72) Inventors: Zhenglun Zhu, Newton, MA (US); Hong Gao, Dowingtown, PA (US)

(21) Appl. No.: 18/918,938

(22) Filed: Oct. 17, 2024

Related U.S. Application Data
(62) Division of application No. 17/675,800, filed on Feb. 18, 2022, now Pat. No. 12,146,138.
Application No. 17/675,800 is a division of application No. 16/075,479, filed on Aug. 3, 2018, now Pat. No. 11,268,095.
(60) Provisional application No. 62/291,271, filed on Feb. 4, 2016.

(51) Int. Cl. C12N 15/113; A61K 31/7115; A61K 35/17; A61K 40/17; A61K 40/19; A61K 40/22; A61K 40/24; A61K 40/41; A61P 29/00; C12N 5/0786; C12Q 1/68; C12Q 1/6883; G01N 33/50; G01N 33/68

(52) U.S. Cl. CPC C12N 15/1137; A61K 31/7115; A61K 35/17; A61K 40/17; A61K 40/19; A61K 40/22; A61K 40/24; A61K 40/416; C12N 5/0645; C12N 15/113; C12Q 1/68; C12Q 1/6883; G01N 33/5023; G01N 33/6875; A61P 29/00; C12N 2310/11; C12N 2310/14; C12N 2310/3233; C12Q 2600/158; G01N 2333/4703; G01N 2800/52; G01N 2800/7095

(57) ABSTRACT

A method of treating an inflammatory disorder in a subject, comprising administering to a subject in need thereof a nucleic acid molecule for inhibiting the expression of Hom-1.

### REFERENCE TO RELATED APPLICATIONS

This application is a divisional application of U.S. application Ser. No. 17/675,800, filed on Feb. 18, 2022, which is a divisional application of U.S. application Ser. No. 16/075,479, filed on Aug. 3, 2018, now U.S. Pat. No. 11,268,095, which is the National Stage of International Application No. PCT/US2017/015775, filed Jan. 31, 2017, which claims the benefit of U.S. Provisional Application No. 62/291,271, filed Feb. 4, 2016. The entire contents of each of the foregoing applications, including sequence listings, are hereby incorporated by reference.

### REFERENCE TO SEQUENCE LISTING

The instant application contains a Sequence Listing which has been submitted electronically in XML format and is hereby incorporated by reference in its entirety. Said XML copy, created on Oct. 15, 2024, is named 130160-00604-SL.xml and is 15,821 bytes in size.

### BACKGROUND

Tissue macrophages play major roles in host defense against pathogen invasion and in homeostasis of immunity. Plasticity is a hallmark of macrophages. Residing in a microenvironment full of signals from host cells and microbial, macrophages can be activated to display pro-inflammatory (M1) phenotype or anti-inflammatory (M2) phenotypes. Aberrant differentiation and activation of macrophages play major roles in pathogenesis of inflammation.

In IBD patients for example, there is an increase of mucosal CD14+ macrophages, which display a M1 pro-inflammatory phenotype. Due to its central executor role of both innate and adaptive immunity, macrophages have been viewed as an ideal target to control autoimmune and inflammatory disorders. However, how macrophage plasticity is regulated remains incompletely understood, and the cell intrinsic factor that can be manipulated to modulate macrophage function remains largely unknown.

### SUMMARY

In one aspect, described below is a method of treating an inflammatory disorder in a subject, comprising administering to a subject in need thereof a nucleic acid molecule for inhibiting the expression of Hom-1.

In one embodiment, the nucleic acid molecule is an RNAi agent or an antisense oligonucleotide. The nucleic acid molecule can be administered topically, orally, rectally, nasally, intravenously, intraarticularly, conjunctivally, intracranially, intraperitoneally, intrapleurally, intramuscularly, intrathecally, or subcutaneously. In one embodiment, the nucleic acid molecule is administered naked.

In another aspect, described herein is a composition for treating an inflammatory disorder, the composition comprising a nucleic acid molecule for inhibiting the expression of Hom-1 and a pharmaceutically acceptable carrier. In one embodiment, the nucleic acid molecule is a morpholino oligonucleotide having the sequence of SEQ ID NO: 3, 4, 5, or 6. The composition can be formulated for topical, oral, rectal, nasal, intravenous, intraarticular, conjunctival, intracranial, intraperitoneal, intrapleural, intramuscular, intrathecal, or subcutaneous route of administration. In one embodiment, the nucleic acid molecule (e.g., a morpholino oligonucleotide having the sequence of SEQ ID NO: 3, 4, 5, or 6) is a naked nucleic acid molecule.

In yet another aspect, a method of identifying a therapeutic for an inflammatory disorder is described. It includes contacting an inflamed tissue sample with a test therapeutic; and detecting the expression level of Hom-1 in the tissue sample. If the expression level is lower than or equal to a control level, the test therapeutic is a candidate therapeutic for the inflammatory disorder.

In one aspect, described below is a method of selecting a therapeutic for an inflammatory disorder in a subject in need thereof, comprising contacting an inflamed tissue sample obtained from the subject with a therapeutic; detecting a lower or same expression level of Hom-1 in the tissue sample as compared to a control level; and administering the therapeutic to the subject.

In another aspect, contemplated herein is a method of monitoring the efficacy of a therapeutic for an inflammatory disorder in a subject in need thereof, comprising detecting the expression level of Hom-1 in an inflamed tissue sample obtained from the subject after the subject has been administered with the therapeutic; comparing the detected level with a control level; and making a treatment decision based on the comparison, wherein, if the detected level is higher than the control level, continue to administer the therapeutic or a different therapeutic to the subject.

In another aspect, a method of treating an inflammatory disorder in a subject in need thereof is described herein. The method includes providing a modified macrophage, monocyte, or dendritic cell that has been treated with a Hom-1 inhibitor or contains an expression construct for expressing a Hom-1 inhibitor, wherein the modified macrophage, monocyte, or dendritic cell expresses a lower level of Hom-1 as compared with a control level; and administering an effective amount of the modified macrophage, monocyte, or dendritic cell to the subject. In one embodiment, the method includes, prior to the providing step, detecting a higher expression of Hom-1 than a control level in an inflamed tissue sample obtained from the subject.

The details of one or more embodiments are set forth in the description below. Other features, objects, and advantages of the embodiments will be apparent from the description and from the claims.

### DETAILED DESCRIPTION

It was unexpectedly discovered that knocking down Hom-1 expression in tissue macrophages can abate tissue inflammation and protect viability of mucosal epithelial cells.

Hom-1, a human homeobox transcriptional factor, is an antagonist of the canonical Wnt signaling. A nucleic acid sequence of Hom-1 (SEQ ID NO: 1) and the amino acid sequence it encodes (SEQ ID NO: 2) are shown below:

(SEQ ID NO: 1; Underlined: the coding sequence)
acctggccgc catgcgcctc tcctcctccc cacctcgtgg
cccgcagcag ctctccagct ttggctccgt ggactggctc
tcccagagca gctgctcagg gccgacccac acccccaggc
ctgccgactt ctccctgggg agcctccctg gcccaggcca
gacatccggc gcccgggagc cccctcaggc cgtcagcatc
aaggaggccg ccgggtcctc aaatctgcct gcgccggaga
ggaccatggc cgggttgagt aaggagccaa ataccttgcg
ggccccccgt gtccgcacag ccttcaccat ggagcaggtc
cgcaccttgg agggcgtctt ccagcaccac cagtacctga
gccctctgga gcggaagagg ctggccaggg agatgcagct
ctcagaggtc cagataaaaa cctggtttca gaatcgccgc
atgaaacaca aacggcaaat gcaggacccc cagctgcaca
gccccttctc ggggtctctc catgcgcccc cagctttcta
ctcaacgtct tctggccttg ccaatggcct gcagctgctg
tgcccttggg cacccctgtc cgggccccag gctctgatgc
tgccccctgg ctccttctgg ggtctctgcc aagtggcaca
agaggccctg gcatctgcgg gagcttcctg ctgcgggcag
cctctggcgt cccacccccc taccccaggc cggccttcgc
tgggaccagc cctgtccacg gggccccggg gcctgtgtgc
tatgccacag acgggggatg cattttgagg aggcacctct
gactcccaca ctcgcggtct tgctgatcgc acctggctcc
tacctggagg actcagttgt tctgtttaca tcctggtggc
acctctcacc ctgacccaca caaaggttct ggagattact
ggagaatata tataaatata tatatgtacg tatatatgta
aatacacata tacgtatata taaatatata tatacatatg
tgtgtgtata tatatatata tttttttttt tttttttttt
tttgagacgg agtgttgctc tgtcacccag gctggagtgc
aatgacgcaa tctcggctca ctgcaacctc cgcctcctgg
gttcaagcga ttctccagcc tcagcctccc gagtagctgg
gattacagac acccgccacc acgcccggct aattttttct
atttttagta gaaatggggt ttcaccatgt tagccaggct
ggtctcaaac tcctgaccct gtgatccgcc cgcctcggcc
tcccaaagtg ctgggattac aggcatgagc cactgcaccc
ggccctgaga atatatttat taaagccacc tcttcactga
aagttaccga aagagtcggt ttaggaagga aacgaagggt
cagtgaacag agtcaaatgc agaagtgggc ttgtcatggg
tagggctttc ggcgtacgat aaaaggatca tttgtttttt
aaaaggggtt ggaaaaactg gttttccagt tggaaacagt
aaaggttgta agctttgtgt gtacaaaaga aaacagggaa
tgcaggtgtg tttatagcgt tgtggttcaa gtccctctta
acaagaactc caaagctgga aagcaggagg gaacaaaggt
gaacatgaag gcgaggatgc tggggccctg cagtgcgctc
taggctgtgc gtgagccggg actgtaccca cagcttgctg
agggctgctc ttcttgggcc agggaaagca gggcagccgg
gacctgcggc tgtgcctgga ctgaagctgt cccgcaggtc
cccaccctcc aacacgtgct cacctgtccc cctcctcgca
gcagcctcgg gacaaaacaa tgactcaagg acagcacttc
tcgcagaagg tctggaagtg cccagaatgg gaggcacgga
agcccctccc ggggaggact cccgcgttga tggaccgttc
ttggtgcaga ctcctgactg cgtgcatgaa acctgagaca
agtgcaattc cttccatgtc gccccagagt gcccaggagg
caggcagtgc ggggtgccca ggcagacggg ttcagcctgc
agaactggag gcgacctgtg aaacccaccc gggcacccca
acaggaacag aagcgtggtc ctgcggctgc gtccccagcg
agtttcactt tccccttgct cgtttctccc ttgttgtaag
tgtttacaac tggcatgtgc ttttaaacgt caggtaagag
gggaacagct gctgtacatc gtcctggcga gtgacaatgt
gacagaagcc tgggcgaggc cctcggaggg cagcagctgg
acaggggcta ctgggtttgg cctggacagc actgatttgt
ggatgtggat gggggcacgt tgtccgtgat aaaagtacaa
gtgcccctca caaaaaaaaa aaaaaaaa
(SEQ ID NO: 2; Underlined: aa. 91-151/homeodomain)
mrlssspprg pqqlssfgsv dwlsqsscsg pthtprpadf
slgslpgpgq tsgareppqa vsikeaagss nlpapertma
glskepntlr aprvrtaftm eqvrtlegvf qhhqylsple
rkrlaremql sevqiktwfq nrrmkhkrqm qdpqlhspfs
gslhappafy stssglangl qllcpwapls gpqalmlppg
sfwglcqvaq ealasagasc cgqplashpp tpgrpslgpa
lstgprglca mpqtgdaf

Described herein is a method of treating an inflammatory disorder in a subject in need thereof by administering to the subject a Hom-1 inhibitor, e.g., a nucleic acid molecule for inhibiting the expression of Hom-1.

The nucleic acid molecule can be an RNAi agent or an antisense oligonucelotide. In one embodiment, the nucleic acid molecule is a morpholino oligonucleotide. A morpholino oligonucleotide has the standard DNA bases (A, C, G, T) but the bases are bound to morpholine rings and linked through phosphorodiamidate groups. An anti-Hom-1 morpholino oligonucleotide can have a sequence selected from

(SEQ ID NO: 3)
5′-TACTCAACCCTGACATAGAGGGTAA-3′,
(SEQ ID NO: 4)
5′-GAGCCCGGTTTGCATACACGGCTAA-3′,
(SEQ ID NO: 5)
5′-GCCCAGATAAGCAGCGCCTAATTGC-3′,
and
(SEQ ID NO: 6)
5′-CTGTAGGAAAAGCAAGATCAGAACA-3′.

The term “RNAi agent” refers to an RNA (or analog thereof), having sufficient sequence complementarity to a target RNA to direct RNA interference. Generally, an interfering RNA (“iRNA”) is a double stranded short-interfering RNA (siRNA) or short hairpin RNA (shRNA) that results in catalytic degradation of specific mRNAs. An antisense oligonucleotide is typically a single-stranded DNA, RNA, or an analog thereof that has a sequence that can bind to a target nucleic acid molecule.

The anti-Hom-1 nucleic acid molecule can be administered to the subject via any route of administration, e.g., topical, oral, rectal, nasal, intravenous, intraarticular, conjunctival, intracranial, intraperitoneal, intrapleural, intramuscular, intrathecal, or subcutaneous route of administration. The route can be selected based on the site of inflammation. A pharmaceutical composition containing an anti-Hom-1 nucleic acid molecule can be formulated for any route of administration, e.g., as an injectable solution, pill, capsule, eye drop, spray, inhaler, topical cream or gel, or aerosol).

In one embodiment, the anti-Hom-1 nucleic acid molecule is administered naked. In other words, no delivery vehicles such as liposomes or viral vectors are used with the nucleic acid molecule.

Prior to the administration of any Hom-1 inhibitor to a subject, the subject can be tested to determine whether he or she has an elevated expression level of Hom-1 and/or an elevated expression level of an inflammatory cytokine as compared to a control level. In one embodiment, the expression level is detected in an inflamed tissue sample obtained from the subject. A subject with an increased expression level of Hom-1 can be treated with a Hom-1 inhibitor. A control level can be a level representative of the Hom-1 expression level in a non-inflamed tissue or subjects without inflammatory disorders, or a level found in a non-inflamed tissue in the subject to be treated.

A “subject” refers to a human and a non-human animal. “Treating” or “treatment” refers to administration of a compound or agent to a subject, who has a disorder, with the purpose to cure, alleviate, relieve, remedy, delay the onset of, or ameliorate the disorder, the symptom of the disorder, the disease state secondary to the disorder, or the predisposition toward the disorder. An “effective amount” refers to an amount of the compound that is capable of producing a medically desirable result in a treated subject. The treatment method can be performed alone or in conjunction with other drugs or therapy.

Also described herein is a screening method of identifying a therapeutic for an inflammatory disorder. The method includes contacting an inflamed tissue sample with a test therapeutic and detecting the expression level of Hom-1 in the tissue sample. If the expression level is lower than or equal to a control level, the test therapeutic is a candidate therapeutic for the inflammatory disorder.

A method of selecting a therapeutic for an inflammatory disorder in a subject in need thereof is also described. The method includes contacting an inflamed tissue sample obtained from the subject with a therapeutic, detecting a lower or same expression level of Hom-1 in the tissue sample as compare to a control level, and administering the therapeutic to the subject.

Further described is a method of monitoring the efficacy of a therapeutic for an inflammatory disorder in a subject in need thereof. The method includes detecting the expression level of Hom-1 in an inflamed tissue sample obtained from the subject after the therapeutic is administered to the subject, comparing the detected level with a control level, and making a treatment decision based on the comparison. If the detected level is lower than the control level, it indicates that the therapeutic is effective for treating inflammation in the subject. If the detected level is the same as or higher than the control level, a decision can be made to continue giving the same therapeutic or to try a different therapeutic.

The therapeutic or test therapeutic can be a protein, peptide, peptidomimetic, peptoid, cell, antibody or fragment thereof, small molecule compound, nucleic acid molecule, or a plant extract. In one embodiment, the therapeutic or test therapeutic can be a steroid, non-steroidal anti-inflammatory drug, or immuno-suppressant.

In the above-described screening, selecting or monitoring method, the control level can be a level representative of the expression level of Hom-1 in a non-inflamed tissue. It can also be the expression level of Hom-1 in the inflamed tissue sample before it was contacted with a therapeutic or test therapeutic. A skilled person would be able to determine suitable control levels.

In one aspect, a method of treating an inflammatory disorder using modified macrophages, monocytes, or dendritic cells is described. The method includes providing modified macrophages, monocytes, or dendritic cells that have been treated with a Hom-1 inhibitor or contain an expression construct for expressing a Hom-1 inhibitor. The modified macrophages, monocytes, or dendritic cells express a lower level of Hom-1 as compared with a control level. An effective amount of the modified macrophages, monocytes, or dendritic cells are administered to a subject with an inflammatory disorder.

The Hom-1 inhibitor can be a protein, peptide, peptidomimetic, peptoid, cell, antibody or fragment thereof, small molecule compound, nucleic acid molecule, or a plant extract. In one embodiment, the inhibitor is an RNAi agent or an antisense oligonucleotide (e.g., a morpholino oligonucleotide).

Prior to administering the modified cells to a subject, the expression level of Hom-1 in a sample (e.g., an inflamed tissue sample) obtained from the subject can be determined. If the expression level is higher than a control level, the subject is deemed as suitable for the treatment. The control level can be a level representative of the level in a non-inflamed tissue or the level detected in a non-inflamed tissue sample obtained from the subject to be treated. Again, a skilled practitioner would be able to determine a suitable control level.

Hom-1 expression level can be determined at either the mRNA level or at the protein level. Methods of measuring mRNA levels and protein levels are well known in the art.

In any of the methods described herein, in addition to or alternatively to detecting the expression level of Hom-1 as an indicator of inflammation (e.g., the presence or degree of inflammation), detecting the expression and/or secretion of a pro-inflammatory cytokine, the expression and/or secretion of an anti-inflammatory cytokine, the expression of a marker of M1 or M2 macrophages, the expression of a marker of DC differentiation and activation can also be used to measure inflammation.

An inflammatory disorder is characterized by a local or systemic, acute, or chronic inflammation. Inflammatory disorders include, but are not limited to, inflammatory dermatoses (e.g., dermatitis, psoriasis, eczema, atopic dermatitis, allergic contact dermatitis, urticaria, necrotizing vasculitis, cutaneous vasculitis, hypersensitivity vasculitis, eosinophilic myositis, polymyositis, dermatomyositis, or eosinophilic fasciitis), inflammatory bowel diseases (e.g., Crohn's disease and ulcerative colitis), acute respiratory distress syndrome, fulminant hepatitis, pancreatitis, hypersensitivity lung diseases (e.g., hypersensitivity pneumonitis, eosinophilic pneumonia, delayed-type hypersensitivity, interstitial lung disease or ILD, idiopathic pulmonary fibrosis, and ILD associated with rheumatoid arthritis), asthma, COPD, allergic rhinitis, rheumatoid arthritis, psoriatic arthritis, systemic lupus erythematosus, myasthenia gravis, juvenile onset diabetes, glomerulonephritis, autoimmune throiditis, ankylosing spondylitis, systemic sclerosis, multiple sclerosis, primary lateral sclerosis, amyotrophic lateral sclerosis, anaphylaxia, systemic anaphylaxia, hypersensitivity responses, systemic inflammatory conditions, drug allergies, insect sting allergies, allograft rejection, graft-versus-host disease, Sjogren's syndrome, human immunodeficiency, a virus infection, atherosclerosis, hypertension, diabetes, and chronic renal diseases, ocular inflammatory diseases, uveitis and conjunctivitis, neuritis.

A skilled practitioner would be able to determine whether a person has an inflammatory disorder. The expression level of Hom-1 in a sample (e.g., a tissue, cell or bodily fluid sample) obtained from a subject suspected of having an inflammatory disorder can also be used as a diagnostic tool.

The specific example below is to be construed as merely illustrative, and not limitative of the remainder of the disclosure in any way whatsoever. Without further elaboration, it is believed that one skilled in the art can, based on the description herein, utilize the present disclosure to its fullest extent.

### EXAMPLE

Macrophages are key regulators of both innate and adaptive immunity. How macrophage plasticity is regulated by cell intrinsic factors is incompletely understood. The data described below demonstrate that the human homeobox transcription factor, Hom-1, plays a pivotal role in directing macrophage polarization towards the M1 phenotype. Hom-1 expression is aberrantly elevated in tissue macrophages isolated from inflamed mucosa of IBD patients. Using an en bloc culture model, we showed that knockdown of Hom-1 expression in tissue macrophages by morpholigo oligonucleotides can abate tissue inflammation and protect viability of mucosal epithelial cells. Taken together, our data suggest that Hom-1 can serve as a novel target to manage inflammatory disorders.

Hom-1 expression is up-regulated in macrophages isolated from inflamed gastrointestinal mucosa

Using an in vitro monocyte-derived macrophage model, we showed that Hom-1 controls monocytes to macrophage differentiation and pro-inflammatory activation. To explore the potential role of Hom-1 in tissue macrophage differentiation and activation, we examined Hom-1 expression in mucosal macrophages isolated from mucosa of IBD patients. We found that Hom-1 expression was significantly elevated in macrophages isolated from inflamed mucosa, in comparison to the control macrophages isolated from normal mucosa of the same patients. Using FACAS and ELISA analysis, we found that, in parallel to the elevated expression of Hom-1, the expression of M1 surface markers, such as CD40, CD80, and CD86 as well as the expression and secretion of M1 pro-inflammatory cytokines were elevated in macrophages isolate from inflamed mucosa. In addition, we found that the expression of reactive oxygen species (ROS) and Nitric oxide (NO) were also elevated in macrophages isolated from inflamed mucosa.

Hom-1 regulates mucosal macrophage plasticity and polarizes mucosal macrophage towards M1 phenotype

Plasticity is a hall mark of macrophages. In response to environmental cues, macrophages display spectrums of phenotypes, ranging from the classic pro-inflammatory M1 phenotype to a variety of M2 phenotypes with distinguished features. Corticosteroids have been used extensively to manage inflammatory disorders and have been shown to induce M2 phenotype of macrophages. To determine whether Hom-1 plays a role in regulating mucosal macrophage plasticity, we examined the effects of Corticosteroids on the expression of Hom-1. Incubation of mucosal CD14 macrophages with predinisolone led to a significant reduced level of Hom-1 expression and a characteristic reduced secretion of M1 cytokines IL12, but an increased secretion of M2 cytokine IL10. Consistent with a potential role of Hom-1 in regulating macrophage plasticity, we found that the morphologies of GFP transfected but not the GFP-Hom-1 transfected mucosal macrophages can be induced by PD to display characteristic roundup phenotypes. FACS analysis of cell surface expression of CD80 and ELISA analysis of secretion of IL12 culture media in GPF or GFP-Hom-1 expressing macrophages showed that Hom-1 rendered the macrophages resistant to PD induced reduction of CD80 and secretion of IL12. To further explore whether Hom-1 regulates macrophage polarization, we examined Hom-1 expression during induced M2 to M1 switch, using the in vitro macrophage differentiation model as previously described. We found that Hom-1 expression is elevated during induced M2 to M1 polarization. To define a key regulatory role of Hom-1 in polarization of macrophages, we examined the effects of knocking-down Hom-1 in LPS-induced M2 to M1 phenotype switch. We found that down-regulation of Hom-1 renders macrophages resistant to the LPS induced M1 polarization, suggesting a key regulatory role of Hom-1 in the process. To further define the function of Hom-1 in macrophage polarization, we explored the effects of ectopic expression of Hom-1 in M2 macrophages and found that over expression of Hom-1 led to a significant increase of surface expression of M1 marker, CD 80 as well as elevated secretion of M1 cytokines, IL1b, IL12 and TNFa. Taken together, the data suggested that Hom-1 plays a key role in regulating macrophages plasticity and polarizes macrophages towards M1 phenotype.

### Hom-1 Differentially Regulates the Expression of M1 and M2 Genes

To explore the potential mechanisms of Hom-1 regulated polarization of macrophages, we examined the effects of ectopic expression of Hom-1 on the expression characteristic M1 and M2 genes. We found that Hom-1 expression promotes the expression of M1 genes, such as IL1, IL12 and TNFa, but suppresses the expression of M2 genes, such as IL10 and TGFb. Together with our previous findings that Hom-1 expression is required for the expression of M1 genes, such not the expression of tested M2 genes, our data suggested that Hom-1 regulates macrophage plasticity through polarizing the expression of M1 and M2 genes.

### Targeting Hom-1-Regulated Macrophage Plasticity in Pathogenesis of Tissue Inflammation

To further determine whether Hom-1-regulated macropahges can be targeted to abate tissue inflammation, we used en bloc culture of tissues obtained from inflamed or normal mucosa of ulcerative colitis (UC) patients. We found that, consistent with clinical findings, the secretion of inflammatory cytokines, such as TNFa, IL1β and Nitrate was significantly elevated in the culture of inflamed tissues. We then added anti-Hom-1 morpholino oligonucleotides (MO) to the tissue cultures and examined whether it can down-regulate the expression of Hom-1 in mucosal macrophages. We found that Hom-1 Mo efficiently inhibited Hom-1 expression in macrophages in the en bloc tissue cultures. To further explore the effect of Hom-1 MO on tissue inflammation, we examined the concentrations of TNFa during the incubation of en bloc tissue with Hom-1, using ELISA assay. We found that Hom-1 MO reduced the amount of TNFa in the cultures in a dosage dependent manner. To further explore the effects of Hom-1 MO on the secretion of other pro-inflammatory cytokines, we examined the effects of Hom-1 MO on the secretion of IL1 and Nitrate and found that, similar to the TNFa, Hom-1 MO exerts strong inhibition of these pro-inflammatory cytokines in the en bloc culture systems. As the ex-vivo en bloc culture may reflect the tissue microenvironment in vivo, our data suggested that Hom-1 MO can target tissue macrophages to abate tissue inflammation.

### Hom-1 MO Rescue Viability of Epithelial Cells in Inflamed Tissue Inflammation

Apoptosis of epithelial cells, which causes mucosal ulceration, is a hallmark of IBD. Tissue inflammation has been thought to be the major trigger of apoptosis of mucosal epithelial cells. Using en bloc tissue culture, we found that there was a greater rate of apoptosis of mucosal epithelial cells in tissues isolated from inflamed mucosa in comparison to the apoptotic rate of epithelial cells in normal control tissue. When Hom-1 MO was added to the en bloc tissue culture, we found that Hom-1 MO but not the control MO exerted strong inhibitory effects on apoptosis of epithelial cells in tissue culture. Taken together with our findings that Hom-1 MO can abate tissue inflammation, the data suggested that Hom-1 MO can function as an agent to manage inflammation.

### Monocytes Isolation and Culture

Peripheral blood mononuclear cells (PBMC) from healthy adult donors at Children's Hospital Boston were isolated by Ficoll density gradient centrifugation. Experiments with human materials were performed in accordance with guidelines approved by the institutional review committee of Brigham and Women's Hospital. CD14+ monocytes were purified from PBMCs using anti-CD14-coated microbeads (Miltenyi Biotec). The purity of freshly isolated CD14+ monocytes was more than 95% as analyzed by flow cytometry. Monocytes were cultured in 12-well plates at 1×10⁶ cells/ml with RPMI 1640 medium containing 10% fetal bovine serum (FBS). M-CSF, GM-CSF, and IL3 were purchased from PeproTech and used at the final concentration of 100 ng/ml. Cytokines were added to cultures every 2 or 3 days.

### RNA Interference

Human primary monocytes were transfected using the Human Monocyte Nucleofector Kit (Lonza) according to the manufacturer's instructions. Briefly, 5×10⁶ monocytes were resuspended into 100 μl nucleofector solution with 0.5 nmol of either Hom-1 siRNA (forward: 5′-UUCAGAAUCGCCGCAUGAAACACAAACGG-3′ (SEQ ID NO: 7); reverse: 5′-CCGUUUGUGUUUCAUGCGGCGAUUCUGAA-3′ (SEQ ID NO: 8)) or non-effective GFP siRNA (forward: 5′-UGACCACCCUGACCUACGGCGUGCAGUGC-3′ (SEQ ID NO: 9); 5′-reverse: GCACUGCACGCCGUAGGUCAGGGUGGUCA-3′ (SEQ ID NO: 10)) before electroporation with nucleofector II Device (Lonza). Cells were then immediately removed from the device and incubated overnight with 1 ml pre-warmed Human Monocyte Nucleofector

Medium containing 2 mM glutamine and 10% FBS. Cells were then resuspended into complete RPMI medium and treated with appropriate cytokines to induce differentiation into macrophages. Similarly, macrophages derived from monocytes were transfected with Human Macrophage Nucleofector Kit (Lonza) following the manufacturer's instructions.

### FACS Analysis

Phenotypic analysis of monocytes/macrophages was performed using flow cytometry after immunolabeling of cells with fluorescence dye conjugated antibodies. The following antibodies were used: PE-conjugated anti-CD71, CD11b, CD11c, CD16, CD64, CD80, CD86, HLA-DR, CD14, TLR4, IL1-B and TNF-a, and FITC-conjugated anti-CD40, CD36 (eBioscience); FITC-conjugated anti-mannose receptor (MR), and unconjugated mouse anti-MCSFR (R&D Systems). Isotope control labeling was performed in parallel. Antibodies were diluted as recommended by the supplier. PE-conjugated rabbit against mouse IgG antibody was used for secondary M-CSFR staining. Labeled cells were analyzed with FACScan flow cytometer (BD Bioscience) using CellQuest software. Results are expressed as the percentage of positive cells and/or mean fluorescence intensity (MFI) values after subtraction of the MFI obtained with the isotype control antibody.

### RT-PCR

Total RNA was isolated by the TRIzol reagent, and an equal amount of RNA was used for first-strand cDNA synthesis with SuperScript III First-Strand Synthesis System (Invitrogen) according to the manufacturer's protocol. To amplify Hom-1 cDNA with conventional PCR, AccuPrime™ Taq DNA polymerase system (Invitrogen) was used following the manufacturer's instructions. PCR products were separated on 2% agarose gels and stained with ethidium bromide. GAPDH was used as an internal control. We performed quantitative measurement of Hom-1 and cytokines cDNA with SYBR Green on a LightCycler® (480 Real-Time PCR System; Roche).

### Cytokine Measurements

Levels of IL-1β and TNF-α and IL12p70 in the supernatants of E. coli LPS (Sigma) and IFN-γ (PeproTech) treated macrophage or LPS treated U937 cells were quantified using ELISA kits obtained from eBiosciences. Analyses were conducted according to the manufacturer's instructions.

### Detection of Reactive Oxygen Species (ROS) and Nitric Oxide (NO)

The ROS level in activated macrophages was detected with Image-iT® LIVE Green Reactive Oxygen Species Detection Kit (Invitrogen) basically following the manufacturer's instructions except that the results were analyzed by both fluorescence microscope and flow cytometry. The NO level was determined by Griess Reagent Kit for Nitrite Determination (Invitrogen) following the protocol provided by the manufacturer.

### Cytostaining

For Wright-Giemsa staining, a staining kit from Sigma was used according to the manufacturer's instructions.

### Statistical Analysis

Data were analyzed using the paired Student's t test (2-tailed) and Wilcoxon rank-sum test. The differences with p value <0.05 were considered statistically significant.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the described embodiments, and without departing from the spirit and scope thereof, can make various changes and modifications of the embodiments to adapt it to various usages and conditions. Thus, other embodiments are also within the claims.

1. A method of treating an inflammatory disorder in a subject, comprising administering to a subject in need thereof a nucleic acid molecule for inhibiting the expression of Hom-1.

2-13. (canceled)

14. A method of identifying a therapeutic for an inflammatory disorder, comprising:
contacting an inflamed tissue sample with a test therapeutic; and
detecting the expression level of Hom-1 in the tissue sample;
if the expression level is lower than or equal to a control level, the test therapeutic is a candidate therapeutic for the inflammatory disorder.

15. (canceled)

16. A method of monitoring the efficacy of a therapeutic for an inflammatory disorder in a subject in need thereof, comprising:
detecting the expression level of Hom-1 in an inflamed tissue sample obtained from the subject after the subject has been administered with the therapeutic;
comparing the detected level with a control level; and
making a treatment decision based on the comparison, wherein, if the detected level is higher than the control level, continue to administer the therapeutic or a different therapeutic to the subject.

17. The method of claim 16, wherein the therapeutic is a steroid, non-steroidal anti-inflammatory drug, immuno-suppressant.

18. The method of claim 16, wherein the therapeutic is a Hom-1 inhibitor.

19. The method of claim 16, wherein the therapeutic is a protein, peptide, peptidomimetic, peptoid, cell, antibody or fragment thereof, small molecule compound, nucleic acid molecule, or a plant extract.

20. The method of claim 16, wherein the control level corresponds to the expression level of Hom-1 in a non-inflamed tissue sample or in the inflamed tissue sample before it was contacted with a therapeutic.

21-24. (canceled)


SEQUENCE LISTING (10 sequences)

SEQ ID NO: 1 - DNA, 2428 bp, Homo sapiens
  source 1..2428: mol_type = other DNA
  CDS 12..788: note = Hom-1
acctggccgccatgcgcctctcctcctccccacctcgtggcccgcagcagctctccagctttggctccgtggactggctctcccagagcagctgctcagggccgacccacacccccaggcctgccgacttctccctggggagcctccctggcccaggccagacatccggcgcccgggagccccctcaggccgtcagcatcaaggaggccgccgggtcctcaaatctgcctgcgccggagaggaccatggccgggttgagtaaggagccaaataccttgcgggccccccgtgtccgcacagccttcaccatggagcaggtccgcaccttggagggcgtcttccagcaccaccagtacctgagccctctggagcggaagaggctggccagggagatgcagctctcagaggtccagataaaaacctggtttcagaatcgccgcatgaaacacaaacggcaaatgcaggacccccagctgcacagccccttctcggggtctctccatgcgcccccagctttctactcaacgtcttctggccttgccaatggcctgcagctgctgtgcccttgggcacccctgtccgggccccaggctctgatgctgccccctggctccttctggggtctctgccaagtggcacaagaggccctggcatctgcgggagcttcctgctgcgggcagcctctggcgtcccacccccctaccccaggccggccttcgctgggaccagccctgtccacggggccccggggcctgtgtgctatgccacagacgggggatgcattttgaggaggcacctctgactcccacactcgcggtcttgctgatcgcacctggctcctacctggaggactcagttgttctgtttacatcctggtggcacctctcaccctgacccacacaaaggttctggagattactggagaatatatataaatatatatatgtacgtatatatgtaaatacacatatacgtatatataaatatatatatacatatgtgtgtgtatatatatatatatttttttttttttttttttttttgagacggagtgttgctctgtcacccaggctggagtgcaatgacgcaatctcggctcactgcaacctccgcctcctgggttcaagcgattctccagcctcagcctcccgagtagctgggattacagacacccgccaccacgcccggctaattttttctatttttagtagaaatggggtttcaccatgttagccaggctggtctcaaactcctgaccctgtgatccgcccgcctcggcctcccaaagtgctgggattacaggcatgagccactgcacccggccctgagaatatatttattaaagccacctcttcactgaaagttaccgaaagagtcggtttaggaaggaaacgaagggtcagtgaacagagtcaaatgcagaagtgggcttgtcatgggtagggctttcggcgtacgataaaaggatcatttgttttttaaaaggggttggaaaaactggttttccagttggaaacagtaaaggttgtaagctttgtgtgtacaaaagaaaacagggaatgcaggtgtgtttatagcgttgtggttcaagtccctcttaacaagaactccaaagctggaaagcaggagggaacaaaggtgaacatgaaggcgaggatgctggggccctgcagtgcgctctaggctgtgcgtgagccgggactgtacccacagcttgctgagggctgctcttcttgggccagggaaagcagggcagccgggacctgcggctgtgcctggactgaagctgtcccgcaggtccccaccctccaacacgtgctcacctgtccccctcctcgcagcagcctcgggacaaaacaatgactcaaggacagcacttctcgcagaaggtctggaagtgcccagaatgggaggcacggaagcccctcccggggaggactcccgcgttgatggaccgttcttggtgcagactcctgactgcgtgcatgaaacctgagacaagtgcaattccttccatgtcgccccagagtgcccaggaggcaggcagtgcggggtgcccaggcagacgggttcagcctgcagaactggaggcgacctgtgaaacccacccgggcaccccaacaggaacagaagcgtggtcctgcggctgcgtccccagcgagtttcactttccccttgctcgtttctcccttgttgtaagtgtttacaactggcatgtgcttttaaacgtcaggtaagaggggaacagctgctgtacatcgtcctggcgagtgacaatgtgacagaagcctgggcgaggccctcggagggcagcagctggacaggggctactgggtttggcctggacagcactgatttgtggatgtggatgggggcacgttgtccgtgataaaagtacaagtgcccctcacaaaaaaaaaaaaaaaaa

SEQ ID NO: 2 - protein, 258 aa, Homo sapiens
MRLSSSPPRGPQQLSSFGSVDWLSQSSCSGPTHTPRPADFSLGSLPGPGQTSGAREPPQAVSIKEAAGSSNLPAPERTMAGLSKEPNTLRAPRVRTAFTMEQVRTLEGVFQHHQYLSPLERKRLAREMQLSEVQIKTWFQNRRMKHKRQMQDPQLHSPFSGSLHAPPAFYSTSSGLANGLQLLCPWAPLSGPQALMLPPGSFWGLCQVAQEALASAGASCCGQPLASHPPTPGRPSLGPALSTGPRGLCAMPQTGDAF

SEQ ID NO: 3 - DNA, 25 bp, synthetic construct
  misc_feature 1..25: note = Synthetic oligonucleotide
  source 1..25: mol_type = other DNA
tactcaaccctgacatagagggtaa

SEQ ID NO: 4 - DNA, 25 bp, synthetic construct
  misc_feature 1..25: note = Synthetic oligonucleotide
  source 1..25: mol_type = other DNA
gagcccggtttgcatacacggctaa

SEQ ID NO: 5 - DNA, 25 bp, synthetic construct
  misc_feature 1..25: note = Synthetic oligonucleotide
  source 1..25: mol_type = other DNA
gcccagataagcagcgcctaattgc

SEQ ID NO: 6 - DNA, 25 bp, synthetic construct
  misc_feature 1..25: note = Synthetic oligonucleotide
  source 1..25: mol_type = other DNA
ctgtaggaaaagcaagatcagaaca

SEQ ID NO: 7 - RNA, 29 nt, synthetic construct
  misc_feature 1..29: note = Synthetic oligonucleotide
  source 1..29: mol_type = other RNA
ttcagaatcgccgcatgaaacacaaacgg

SEQ ID NO: 8 - RNA, 29 nt, synthetic construct
  misc_feature 1..29: note = Synthetic oligonucleotide
  source 1..29: mol_type = other RNA
ccgtttgtgtttcatgcggcgattctgaa

SEQ ID NO: 9 - RNA, 29 nt, synthetic construct
  misc_feature 1..29: note = Synthetic oligonucleotide
  source 1..29: mol_type = other RNA
tgaccaccctgacctacggcgtgcagtgc

SEQ ID NO: 10 - RNA, 29 nt, synthetic construct
  misc_feature 1..29: note = Synthetic oligonucleotide
  source 1..29: mol_type = other RNA
gcactgcacgccgtaggtcagggtggtca